(19) United States
(12) Patent Application Publication
(10) Pub. No.: US 2009/0083866 A1
(43) Pub. Date: Mar. 26, 2009

(54) TRANSGENIC ROSA26-LUCIFERASE MICE FOR BIOLUMINESCENT IMAGING

(76) Inventors: Zhenyu Gu, Shanghai (CN); Mary Cole, San Francisco, CA (US)

Correspondence Address: GENENTECH, INC., 1 DNA WAY, SOUTH SAN FRANCISCO, CA, 94080, US

(21) Appl. No.: 12/090,003

(22) PCT Filed: Oct. 10, 2006
(86) PCT No.: PCT/US2006/039035

§ 371 Date: Sep. 17, 2008

Related U.S. Application Data
(60) Provisional application No. 60/726,521, filed on Oct. 13, 2005.

(51) Int. Cl. G01N 33/48; A01K 67/027; A61K 49/00

(52) U.S. Cl. 800/3; 800/13; 424/9.1

(57) ABSTRACT

The invention relates to non-human transgenic animals expressing bioluminescent markers

The invention provides a transgenic mouse whose genome comprises a nucleotide sequence encoding lucif erase, operably linked to murine control sequences whereby the lucif erase is expressed in all cells. Administration of a luciferin substrate to the mouse results in bioluminescence. The control sequence is the Rosa 26 promoter. This mouse is useful to monitor in situ cell growth, differentiation or proliferation

This application claims the benefit of U.S. Provisional Application No. 60/726,521, filed Oct. 13, 2005, the disclosure of which is incorporated by reference herein in its entirety.

### FIELD OF THE INVENTION

The present invention relates to transgenic animal models and imaging methods using the same.

### BACKGROUND OF THE INVENTION

The transgenic mouse model has proven itself to be a useful tool in the discovery of gene function, cell function or organogenesis. A transgene is an introduced DNA sequence which becomes integrated into the genome of a cell from which a transgenic animal develops. Typically DNA sequences are used to generate transgenic animals that express a particular protein at a time and/or place where it is not normally expressed. Transgenic mice can be created that express the gene throughout all stages of development or life of the animal, or in quantities much higher than is found in the normal animal. Methods for generating transgenic animals, particularly animals such as mice or rats, have become conventional in the art and are described, for example, in U.S. Pat. Nos. 4,736,866 and 4,870,009. For example, particular cells may be targeted for transgene expression with tissue-specific control sequences. Transgenic animals that include a copy of a transgene incorporated into the germ line of the animal can be used to crossbreed with other animals of certain genetic backgrounds to further define a pathway or disease. Such animals can be used as tester animals for agents to confer protection from pathological conditions associated with transgene overexpression. An animal is treated with the agent and a reduced incidence of the pathological condition, compared to untreated transgenic animals would indicate a potential therapeutic intervention for the pathological condition.

Transgenics are distinguished from “knock out” animals which have a defective or altered gene as a result of homologous recombination between the endogenous gene and laboratory-altered genomic DNA. Typically, several kilobases of unaltered flanking DNA (both at the 5′ and 3′ ends) are included in the vector [see e.g., Thomas and Capecchi, Cell, 51:503 (1987) for a description of homologous recombination vectors]. When this is introduced into an embryonic stem cell of the animal, the endogenous gene is ablated or “knocked out” in contrast to transgenics which usually promote the expression of a particular gene.

The use of transgenic animals have shed light on particular gene or cell function, but one of the major drawbacks is that the animal must often be sacrificed to examine this function. This often leads to seeing only the later occurring defects in gene or cell function, when the transgenic animal displays a pathological phenotype. Another drawback is the sacrifice of the animal may lead to decreasing numbers of experimental time points. A study must begin with a large number of animals to take a statistically significant number at each experimental time point. This is expensive as large number of animals must be bred, housed and fed during the course of the experiment. It is also time consuming to breed and analyze the animals used for each experiment. A further drawback is embryonic lethality. When certain genes are perturbed, this can result in embryonic lethality. This may lead to few or no transgenic progeny, and the investigator must determine at what embryonic stage the pathology is occurring.

To address this problem, control sequences fused to marker genes which can track the development of cells or organs are useful. Two markers which have proven beneficial are Green Fluorescence Protein (GFP) or the firefly luciferase gene. Researchers have described how transgenic animals expressing these marker genes can be imaged continuously to assay gene expression, analyze tumor growth, determine cell lineage, or follow the progress of infections (Contag et al., J. of Mag. Res. Imag. 16:378-387 (2002)). In general, fluorescence markers such as GFP are easier to use, and can be implemented using common laboratory camera systems and fluorescence microscopes. Whole transgenic animal fluorescent imaging is complicated by the difficulty of directing the necessary excitation light into the transgenic animals and is restricted by tissues that have a certain quantity of autofluoresence which increases the signal to noise ratio (S/N). In contrast, bioluminescence imaging based on luciferase requires administration of the enzyme substrate luciferin that reaches target cells via the blood and diffusion. Because luciferase is not expressed in mammals, the S/N ratio is much cleaner, and autofluoresence is no longer an issue.

Despite the above identified advances in transgenic animal research, there is a great need for additional models capable of expressing luciferase consistently and at high enough levels to be imaged. Previous attempts to prepare transgenic mice expressing luciferase have not achieved expression of luciferase in all cell types or at sufficient levels due to lack of incorporation of effective control sequences into the transgene. Here, a luciferase transgenic animal, wherein expression of luciferase is facilitated by the Rosa26 promoter which expresses luciferase at high levels throughout all tissues is described. In addition, tissues or cells derived from luciferase positive transgenic animals can be xenografted into normal animals and the growth, differentiation and proliferation of the xenografted cells can be monitored and quantified. Therefore, transgenic animals which can be imaged at the whole animal level, tracking time dependent events such as development, cancer growth, circadian rhythms and disease, fulfills a long felt need in the art.

### SUMMARY OF THE INVENTION

The present invention generally relates to non-naturally occurring non-human transgenic animals expressing bioluminescent markers.

The invention provides a transgenic animal whose genome comprises a nucleotide sequence encoding luciferase. The nucleotide sequence is preferably operably linked to murine control sequences whereby the luciferase is expressed in all cells of the mouse. Administration of a luciferin substrate to the transgenic animal results in bioluminescence that can be visualized. Preferably, the control sequence is the Rosa26 promoter. In one embodiment, the Rosa26 promoter comprises the sequence of SEQ ID NO:1. In another embodiment, the Rosa26 promoter consists essentially of the sequence of SEQ ID NO:1. In yet another embodiment, the Rosa26 promoter comprises a comparably active fragment of the sequence of SEQ ID NO:1. A “comparably active fragment” means a fragment that drives expression of an operably linked nucleic acid to a level that is at least about 75%, 80%, 85%, 90%, 95%, or 100% of the level of expression that would result if the nucleic acid were operably linked to a Rosa26 promoter consisting of SEQ ID NO:1.

The present invention further provides a transgenic animal useful as a model for any experiment where gene expression or cell function needs to be assayed without necessarily destroying the animal or cells. Alternatively, the transgenic animal is useful to monitor in situ cell growth, differentiation or proliferation in the animal without necessarily destroying the transgenic animal. Furthermore, the invention provides methods useful to monitor live cell growth, differentiation or proliferation of the transgenic animal by bioluminescent imaging.

In another embodiment the invention provides crossing the luciferase transgenic animals with other transgenic animals. Specifically, luciferase transgenic mice can be crossed with mice containing a MMTV-HER2 transgene, and the offspring assayed for tumor growth and metastasis. Furthermore, offspring from crossed mice can be used as a tissue source to xenograft tissue that is luciferase positive into other disease model mice, and the luciferase positive tissue graft monitored for growth and metastasis.

In another embodiment the luciferase transgenic animals can be used as a source for bone marrow. Specifically, the bone marrow from luciferase transgenic animals can be transplanted into either lethally irradiated or sub-lethally irradiated mice and the number and distribution of the transplanted cells monitored.

In another embodiment the invention provides for tracking luciferase positive cells in a disease models including for example, multiple sclerosis (MS). Specifically, subsets of immune cells can be isolated from the luciferase transgenic animals and transferred to other mouse models, for example the model of Experimental Autoimmune Encephalomyelitis (EAE) used to study MS.

A further embodiment provides methods of identifying agents capable of treating immune cell disorders. The method comprises isolating immune cells from luciferase transgenic animals and transplanting them into a normal host mouse, thus forming a chimeric mouse where only the immune cells will bioluminesce. Agents are administered to the chimeric mice and the number of immune cells assayed to determine if there is a reduction or proliferation as expected from the agent.

A further embodiment provides methods of identifying agents capable of treating B cell lymphoma. The method comprises isolating B cells from luciferase transgenic animals and transplanting them into a normal host mouse, thus forming a chimeric mouse where only the B cells will bioluminesce. Agents (e.g.anti-CD 20 antibodies) are administered to the chimeric mice and the number of B cells assayed to determine if there is a reduction.

In another embodiment, the transgenic animal comprises a luciferase transgene, where the luciferase transgene is used as disrupting or “knock in” sequence.

In a further embodiment, the animals of the present invention are also useful for assessing toxicity by administration of therapeutics to the luciferase transgenic animals. Treatment specificity, toxicity and efficacy can also be determined by comparison of the therapeutic agent's effect with that in a normal animal or untreated transgenic animal. For example, in one embodiment, a method of testing toxicity of an agent is provided, the method comprising: a) measuring the level of luciferase produced by a transgenic animal expressing luciferase; b) administering the agent to the transgenic animal; and c) imaging the transgenic animal for reduction of luciferase in tissues of the transgenic animal, wherein reduction of luciferase expression is indicative of toxicity.

### BRIEF DESCRIPTION OF THE FIGURES

FIG. 1 shows bioluminescence of ES cells transfected with the Rosa26-luciferase construct.

FIG. 2 shows bioluminescence of Rosa26-luciferase transgenic embryos in utero.

FIG. 3 shows bioluminescence of Rosa26-luciferase transgenic mice and individual dissected organs.

FIG. 4 shows quantitation of luciferase activity of embryos or organs of Rosa26-luciferase positive mice.

FIG. 5 shows a bioluminescent signal from a Rosa26-luciferase transgenic mouse crossed with a MMTV-HER2 transgenic mouse.

FIG. 6 shows the bioluminescent signal of embryos in utero and newborn Rosa26-luciferase heterozygous mice.

FIG. 7 shows a schematic of hematopoietic stem cell repopulation with Rosa26-luciferase transgenic mice.

FIG. 8 shows sublethally irradiated mice two weeks after bone marrow transplantation with bone marrow from Rosa26-luciferase transgenic mice.

FIG. 9 shows sublethally irradiated mice four weeks after bone marrow transplantation with bone marrow from Rosa26-luciferase transgenic mice.

FIG. 10 shows lethally irradiated mice ten days after transplantation with bone marrow from Rosa26-luciferase transgenic mice.

FIG. 11 shows lethally irradiated mice four weeks after transplantation with bone marrow from Rosa26-luciferase transgenic mice.

FIG. 12 shows Rosa26-luciferase chimeric mice treated with anti-CD4 antibody

FIG. 13 shows the T cell distribution of cells expressing CD4 and CD8 antigens.

FIG. 14 shows Rosa26-luciferase chimeric mice treated with anti-BR3 antibody.

FIG. 15 shows Rosa26-luciferase chimeric mice treated with anti-CD4 and anti-BR3 antibodies.

FIG. 16 shows a mouse xenograft model for the analysis of tumor growth.

FIG. 17 shows a mouse EAE model for the analysis of monocytes.

FIG. 18 shows clearance of B cells by treatment with anti-CD20 antibody.

FIG. 19 shows the role of bone marrow derived cells resistant to anti-VEGF antibody treatment.

FIG. 20-22 show bioluminescence of organs dissected from mice lethally irradiated and transplanted with bone marrow from Rosa26-luciferase transgenic mice. The bioluminescent areas are indicative that bone marrow progenitor cells were able to contribute to the cell population in that tissue.

FIG. 23 shows bioluminescence of host animals in which bioluminescent tumors (obtained using the scheme shown in FIG. 16) have been grafted.

### DETAILED DESCRIPTION OF THE PREFERRED EMBODIMENTS

### Definitions

As used herein, the term “transgene” means a nucleic acid sequence (e.g. encoding luciferase) that has been introduced into a cell by way of human intervention such as the described methods herein. A transgene could be partly or entirely heterologous, i.e., foreign, to the transgenic animal or cell into which it is introduced. A transgene can include one or more control sequences and any other nucleic acid, such as introns, that may be necessary for optimal expression of a selected nucleic acid.

The term “heterologous” when used in conjunction with polypeptide or gene refers to a polypeptide having an amino acid sequence or a DNA encoding the polypeptide that is not found in the transgenic animal. Thus, a transgenic mouse comprising a firefly luciferase gene can be described as having a heterologous luciferase gene. The transgene can be detected using a variety of methods including PCR, Western blot, or Southern blot.

The term “construct” refers to a nucleic acid vector comprising a heterologous nucleic acid sequence, and allows for replication of the entire nucleic acid sequence.

A “targeting construct” refers to a nucleic acid vector comprising a targeting region. A targeting region comprises a sequence that is substantially homologous to an endogenous sequence in a target tissue, cell or animal and that provides for integration of the targeting construct into the genome of the target tissue, cell or animal. Typically, the targeting construct will also include a gene or a nucleic acid sequence of particular interest, a marker gene and appropriate control sequences.

“Disruption” of a gene occurs when a fragment of DNA locates and recombines with an endogenous homologous sequence. Sequence disruptions or modifications may include insertions, missense, frameshift, deletion, or substitutions, or replacements of DNA sequence, or any combination thereof.

“Insertions” include the insertion of heterologous nucleic acid, which may be of animal, plant, fungal, insect, prokaryotic, or viral origin. Insertion, for example, can alter a gene product by inhibiting its production partially or completely or by enhancing a gene product's activity.

The term “endogenous loci” means a naturally occurring genetic loci found in the host animal.

The term “endogenous promoter” refers to a promoter that is naturally associated with a polynucleotide sequence that encodes a native protein.

The term “Rosa26” or “Rosa26 promoter” refers to the murine promoter described in Zambrowicz et al., Proc. Nat. Acad. Sci. 94:3789-94 (1997), and functional portions thereof.

The term “naturally-occurring” or “naturally associated” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

For nucleic acids, the term “substantial homology” indicates that two nucleic acids, or designated sequences thereof, when optimally aligned and compared with; appropriate nucleotide insertions or deletions have at least about 80% sequence identity, more preferably about 81 % sequence identity, more preferably about 82% sequence identity, more preferably about 83% sequence identity, more preferably about 84% sequence identity, more preferably about 85% sequence identity, more preferably about 86% sequence identity, more preferably about 87% sequence identity, more preferably about 88% sequence identity, more preferably about 89% sequence identity, more preferably about 90% sequence identity, more preferably about 91% sequence identity, more preferably about 92% sequence identity, more preferably about 93% sequence identity, more preferably about 94% sequence identity, more preferably about 95% sequence identity, more preferably about 96% sequence identity, more preferably about 97% sequence identity, more preferably about 98% sequence identity, and more preferably about 99% sequence identity to one another.

Methods of aligning two sequences and identifying % identity are known to those of skill in the art. Several computer programs are available for determining % identity. Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared.

Alternatively, substantial homology exists when the segments will hybridize under stringent hybridization conditions, to the complement of the strand. “Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μ/ml), 0.1% SDS, and 10% dextran sulfate at 42° C., with washes at 42° C. in 0.2×SSC (sodium chloride/sodium citrate) followed by a high-stringency wash consisting of 0.1×SSC containing EDTA at 55° C.

The term “epitope tagged” when used herein refers to a chimeric polypeptide comprising a polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

An “isolated” polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal location different from that of natural cells.

“Control sequences” refers to polynucleotide sequences, such as initiation signals, enhancers, and promoters. In preferred embodiments, transcription of the transgene is under the control of a promoter sequence (or other transcriptional regulatory sequence), which controls the expression of the recombinant gene in a cell type in which expression is intended. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation. Generally, but not always, “operably linked” means that the DNA sequences being linked are contiguous, and, in the case of a secretory leader, contiguous and in reading phase. However, enhancers do not have to be contiguous. Linking may be accomplished by ligation at convenient restriction sites. If such sites do not exist, synthetic oligonucleotide adaptors or linkers may be used in accordance with conventional practice.

“Active” or “activity” for the purposes herein refers to form(s) of a polypeptide which retain a biological and/or an immunological activity of native or naturally-occurring polypeptide, wherein “biological” activity refers to a biological function (either inhibitory or stimulatory) caused by a native or naturally-occurring polypeptide other than the ability to induce the production of an antibody against an antigenic epitope possessed by a native or naturally-occurring polypeptide.

The term “antagonist” is used in the broadest sense, and includes any molecule that partially or fully blocks, inhibits, or neutralizes a biological activity of a native polypeptide. In a similar manner, the term “agonist” is used in the broadest sense and includes any molecule that mimics a biological activity of a native polypeptide. Suitable agonist or antagonist molecules specifically include agonist or antagonist antibodies or antibody fragments, fragments or amino acid sequence variants of native polypeptides, peptides, antisense oligonucleotides, small organic molecules, etc. Methods for identifying agonists or antagonists of a polypeptide may comprise contacting a polypeptide with a candidate agonist or antagonist molecule and measuring a detectable change in one or more biological activities normally associated with the polypeptide.

“Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Animal” refers to any organism classified as a mammal, including domestic and farm animals, and zoo, sports, or pet animals, such as dogs, cats, cattle, horses, sheep, pigs, goats, rabbits, etc. Preferably, the animal is a mouse.

Administration “in combination with” one or more further therapeutic agents includes simultaneous (concurrent) and consecutive administration in any order.

“Transgenic animal” or “Tg+” are used interchangeably and are intended to include any animal in which one or more of the cells of the animal contain heterologous nucleic acid encoding luciferase that has been introduced by way of human intervention, such as by laboratory techniques well known in the art. The nucleic acid may be introduced into the cell, directly or indirectly by way of transfection, electroporation, microinjection or by infection with a recombinant virus. This nucleic acid may become integrated within a chromosome, or it may remain as extrachromosomally replicating DNA. The term “Tg+” includes animals that are heterozygous and/or homozygous for luciferase.

“Bioluminescence” refers to light emitted during a chemical reaction in a biological system. For example, bioluminescence the light emitted upon the cleavage of luciferin substrate by luciferase.

“Real time” refers to monitoring any reaction which can be monitored at the actual time the reaction takes place.

“Luciferin” means any substrate which can be enzymatically cleaved by luciferase and result in bioluminescence.

“Knock-out” refers to an animal in which an endogenous gene has been ablated through homologous recombination techniques.

“Knock-in” refers to an animal in which an endogenous gene has been disrupted by the addition of a heterologous sequence. The heterologous sequence can comprise any sequence, but often a functional marker gene is inserted and it is expressed in the same temporal and spatial order as the endogenous gene.

### COMPOSITIONS AND METHODS OF THE INVENTION

### A. Selection and Use of Nucleic Acid Vectors and Host Cells

In general, a nucleic acid molecule encoding a polypeptide of the invention is inserted into a vector, preferably a nucleic acid vector, in order to express the polypeptide in a suitable host cell. These nucleic acid constructs may also be useful to prepare transgenic mice or targeting vectors for knockout or knock-in animals. The nucleic acid vectors may also comprise regulatory nucleic acid sequences operably linked to nucleic acid sequences encoding luciferase. The luciferase may be firefly or Renilla luciferase

Operably linked control sequences usually increase expression of the nucleic acid segment or sequence in a desired cell type. Preferably, these control sequences are genomic in origin. For example, the nucleic acid vector can include control sequences located in the 5′-flanking regions of a gene operably linked to luciferase coding sequences in a manner capable of replicating and expressing the gene in a host cell. Specifically, the control sequences comprise the promoter sequence Rosa26. In some cases, the promoters may provide for tissue specific expression at a level similar to that level of expression in the animal from which the sequence is derived. If additional flanking sequences are useful in optimizing expression, such sequences can be ligated into the nucleic acid vector. Additional sequences for processing or expression of the transgene can be derived from genomic sequences. Optionally, the nucleic acid vector includes a 5′ untranslated region between the promoter and the DNA sequence encoding luciferase. Preferably, the control sequences provide for expression of the luciferase transgene in all cells and at a level so that expression can be detected using standard methodologies such as detection with antibodies, bioluminescence or nucleic acid probes.

A nucleic acid vector encoding a luciferase transgene as described herein can also include a 3′ untranslated region downstream of the DNA sequence. Such regions can stabilize the RNA transcript of the expression system and thus increases the yield of desired protein from the expression system. Among the 3′ untranslated regions useful in the constructs of this invention are sequences that provide a polyA signal. Such sequences may be derived, e.g., from the SV40 small T antigen, or other 3′ untranslated sequences well known in the art. Such untranslated regions can be from the same control region from which the gene is taken or can be from a different gene, e.g., they may be derived from other synthetic, semi-synthetic or natural sources.

In addition, other promoters or other control sequences may be utilized. For example, heterologous promoters may provide for enhanced levels of expression or tissue specific expression. Various promoters having different strengths may be utilized as long the promoter functions in the transgenic animal or in the desired tissue type.

The various methods employed in the preparation of the nucleic acid vectors and transformation of host organisms are known in the art. Host cells are transfected or transformed with expression or cloning vectors described herein for luciferase production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyomithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salinonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomionas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA ; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan^{r}; E. coli W3110 strain 37D6, which has the complete genotype tonA ptr3 phoA E15 (argF-lac)169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for vectors described herein. Saccharomyces cerevisiae is a commonly used lower eukaryotic host microorganism. Others include Schizosacclaromyces pombe (Beach and Nurse, Nature, 290: 140 [1981]; EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 154(2):737-742 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida; Trichoderina reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81: 1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated luciferase are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/-DHFR (CHO, Urlaub and Chasin, Proc. Natl. Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

If a targeted “knock-out” or “knock-in” is desired, a targeting construct can be made. The targeting construct may be produced using standard methods known in the art (see, e.g., Sambrook et al., 1989, Molecular Cloning: A Laboratory Manual, Second Edition, Cold Spring Harbor Laboratory Press, Cold Spring Harbor, N.Y.; E. N. Glover (eds.), 1985, DNA Cloning: A Practical Approach, Volumes I and II; M. J. Gait (ed.), 1984, Oligonucleotide Synthesis; B. D. Hames & S. J. Higgins (eds.), 1985, Nucleic Acid Hybridization; B. D. Hames & S. J. Higgins (eds.), 1984, Transcription and Translation; R. I. Freshney (ed.), 1986, Animal Cell Culture; Immobilized Cells and Enzymes, IRL Press, 1986; B. Perbal, 1984, A Practical Guide To Molecular Cloning; F. M. Ausubel et al., 1994, Current Protocols in Molecular Biology, John Wiley & Sons, Inc.). For example, the targeting construct may be prepared in accordance with conventional ways, where sequences may be synthesized, isolated from natural sources, manipulated, cloned, ligated, subjected to in vitro mutagenesis, primer repair, or the like. At various stages, the joined sequences may be cloned, and analyzed by restriction analysis, sequencing, or the like.

For example, the targeting DNA may be produced by chemical synthesis of oligonucleotides, nick-translation of a double-stranded DNA template, polymerase chain-reaction amplification of a sequence, purification of prokaryotic or target cloning vectors harboring a sequence of interest (e.g., a cloned cDNA or genomic DNA, synthetic DNA or from any of the aforementioned combination) such as plasmids, phagemids, YACs, cosmids, BACs, bacteriophage DNA, other viral DNA or replication intermediates, or purified restriction fragments thereof, as well as other sources of single and double-stranded polynucleotides having a desired nucleotide sequence. Moreover, the length of homology may be selected using known methods in the art. For example, selection may be based on the sequence composition and complexity of the predetermined endogenous target DNA sequence(s).

In one embodiment, the targeting construct of the present invention comprises a targeting region, which comprises a first sequence homologous to a portion or region of the gene to be disrupted and a second sequence homologous to a second portion or region of the gene. The targeting construct may further comprise a positive selection marker, which is preferably positioned between the first and the second DNA sequences. The positive selection marker may be operatively linked to a promoter and a polyadenylation signal.

In another embodiment, the targeting construct may contain more than one selectable maker gene, including a negative selectable marker, such as the herpes simplex virus tk (HSV-tk) gene, which is preferably positioned outside one or both of the homologous arms of the targeting construct. The negative selectable marker may be operatively linked to a promoter and a polyadenylation signal (see, e.g., U.S. Pat. Nos. 5,464,764; 5, 487,992; 5,627,059 and 5,631,153).

### B. Production of Transgenic Animals

Methods for generating transgenic animals of the present invention, including knock-outs and knock-ins, are well known in the art (see generally, Gene Targeting: A Practical Approach, Joyner, ea., Oxford University Press, Inc. (2000)).

The specific line(s) of any animal used to practice this invention are selected for general good health, good embryo yields, good pronuclear visibility in the embryo, and good reproductive fitness. When transgenic mice are to be produced, strains such as C57BL/6 or C57BL/6×DBA/2 Fit, or FVB lines are often used (obtained commercially from Charles River Labs, Boston, Mass., The Jackson Laboratory, Bar Harbor, Me., or Taconic Labs.). Preferred strains are those with H 2b, H-26 or H-2q haplotypes such as C57BL/6 or DBA/1.

Once an appropriate targeting construct has been prepared, the targeting construct may be introduced into an appropriate host cell using any method known in the art. Various techniques may be employed in the present invention, including, for example: pronuclear microinjection; retrovirus mediated gene transfer into germ lines; gene targeting in embryonic stem cells; electroporation of embryos; sperm mediated gene transfer; and calcium phosphate/DNA co-precipitates, microinjection of DNA into the nucleus, bacterial protoplast fusion with intact cells, transfection, polycations, e.g., polybrene, polyornithine, etc., or the like (see, e.g., U.S. Pat. No. 4,873,191; Van der Putten et al., 1985, Proc. Natl. Acad. Sci., USA 82:6148-6152; Thompson et al., 1989, Cell 56:313-321; Lo, 1983, Mol Cell. Biol. 3:1803-1814; Lavitrano et al., 1989, Cell, 57:717-723).

For the purpose of the present invention, transgenic animals include those that carry the transgene only in part of their cells (“mosaic animals”). The transgene can be integrated either as a single transgene, or in concatamers, e.g., head-to-head or head-to-tail tandems. Selective introduction of a transgene into a particular cell type is also possible by following, for example, the technique of Lasko et al., Proc. Natl. Acad. Sci. USA 89, 6232-636 (1992).

Microinjection is a preferred way of creating transgenic animals. Microinjection is preferred for adding genes to the genome of the animal. A general means of producing a transgenic animal by microinjection is to mate female mice and remove fertilized gametes from their oviducts. The gametes are kept in a medium such as M2 medium to maintain their viability (Hogan, B. et al. 1986). A purified nucleic acid vector which includes the sequence to be added to the mouse is prepared and diluted into a buffered solution. At an appropriate concentration, the vector is loaded into a microinjection needle and the gamete to be injected is placed in a microscope chamber where it can be manipulated. The needle is inserted into the male pronucleus of the egg, and the vector solution is injected. The injected egg is then transferred into the oviduct of a pseudopregnant mouse (a mouse stimulated by the appropriate hormones to maintain pregnancy but which is not actually pregnant), where it proceeds to the uterus, implants, and develops to term.

Alternatively, transgenic animals can be created by transgene introduction into an embryonic stem (ES) cell. Transgenes can be efficiently introduced into the ES cells by DNA transfection or by retrovirus-mediated transduction. Such transformed ES cells can thereafter be combined with blastocysts which thereafter colonize the embryo and contribute to the germ line of the resulting chimeric animal.

Retroviral infection can also be used to introduce a transgene into an animal. The developing animal embryo can be cultured in vitro to the blastocyst stage. During this time, the blastomeres can be targets for retroviral infection (Jacnich, R. (1976) PNAS 73:1260-1264). Efficient infection of the blastomeres is obtained by enzymatic treatment to remove the zone pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus- producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

In one embodiment, generation of the transgenic mice may optionally involve disruption of the genetic loci of the transgenic animal and introduction of luciferase into the transgenic animal's genome, at a location specified by the investigator. Inactivation of the endogenous loci is achieved by targeted disruption through homologous recombination in embryonic stem cells. Alternatively, integration of the luciferase construct can occur at any point in the transgenic animal's genome.

Any cell type capable of homologous recombination may be used in the practice of the present invention. Examples of such target cells include cells derived from vertebrates including mammals such as bovine species, ovine species, murine species, simian species, and other eukaryotic organisms such as filamentous fungi, and higher multicellular organisms such as plants.

Preferred cell types include ES cells, which are typically obtained from pre-implantation embryos cultured in vitro (see, e.g., Evans, M. J. et al., 1981, Nature 292:154-156; Bradley, M. O. et al., 1984, Nature 309:255-258; Gossler et al., 1986, Proc. Natl. Acad. Sci. USA 83:9065-9069; and Robertson et al., I 1986, Nature 322:445-448). The ES cells are cultured and prepared for introduction of the targeting construct using methods well known to the skilled artisan. (see, e.g., Robertson, E. J. ed. “Teratocarcinomas and Embryonic Stem Cells, a Practical Approach”, IRL Press, Washington D.C., 1987; Bradley et al., 1986, Current Topics in Devel. Biol. 20:357-371; by Hogan et al., in “Manipulating the Mouse Embryo”: A Laboratory Manual, Cold Spring Harbor Laboratory Press, Cold Spring Harbor N.Y., 1986; Thomas et al., 1987, Cell 51:503; Koller et al., 1991, Proc. Natl. Acad. I Sci. USA, 88:10730; Dorin et al., 1992, Transgenic Res. 1:101; and Veis et al., 1993, Cell 75:229). The ES cells that will be inserted with the targeting construct are derived from an embryo or blastocyst of the same species as the developing embryo into which they are to be introduced. ES cells are typically selected for their ability to integrate into the inner cell mass and contribute to the germ line of an individual when introduced into an embryo at the blastocyst stage of development. Thus, any ES cell line having this capability is suitable for use in the practice of the present invention.

After the targeting construct has been introduced into cells, the cells in which successful gene targeting has occurred are identified. Insertion of the targeting construct into the targeted gene is typically detected by identifying cells that express the marker gene. In a preferred embodiment, the cells transformed with the targeting construct of the present invention are subjected to treatment with an appropriate agent that selects against cells not expressing the selectable marker. Only those cells expressing the selectable marker gene survive and/or grow under certain conditions. For example, cells that express an introduced neomycin resistance gene are resistant to the compound G418, while cells that do not express the neo gene marker are killed by G418. If the targeting construct also comprises a screening marker such as GFP, homologous recombination can be identified through screening cell colonies under a fluorescent light. Cells that have undergone homologous recombination will have deleted the GFP gene and will not fluoresce. In another example, cells expressing luciferase can be treated with luciferin and sorted for bioluminescence via flow cytometry.

After DNA transfection, ES cell clones carrying the targeted gene can be determined by Southern blot analysis. Cells of one ES cell clone can be injected into blastocysts that can be transferred into foster mothers. Highly chimeric male offspring (80-100% according to coat color) can be bred with C57BL/6 (B6) females for transmitting the transgene to their progeny. Mice homozygous for disruption of the endogenous gene can be obtained at the expected Menedelian frequency by crossing heterozygous offspring.

Alternatively, a positive-negative selection technique may be used to select homologous recombinants. This technique involves a process in which a first drug is added to the cell population, for example, a neomycin-like drug to select for growth of transfected cells, i.e. positive selection. A second drug, such as FIAU, is subsequently added to kill cells that express the negative selection marker, i.e. negative selection. Cells that contain and express the negative selection marker are killed by a selecting agent, whereas cells that do not contain and express the negative selection marker survive. For example, cells with non-homologous insertion of the construct express HSV thymidine kinase and therefore are sensitive to the herpes drugs such as gancyclovir (GANC) or FIAU (1-(2-deoxy 2-fluoro-B D-arabinofluranosyl)-5-iodouracil). (see, e.g., Mansour et al., Nature 336:348-352: (1988); Capecchi, Science 244:1288-1292, (1989); Capecchi, Trends in Genet. 5:70 76 (1989)). Other methods include regulated positive selection (see U.S. 20030032175A1), which requires the addition of a single selective agent.

Successful homologous recombination or insertion of the transgene may be identified by analyzing the DNA of the selected cells to confirm the presence of the heterologous DNA. Various techniques known in the art, such as PCR and/or Southern analysis may be used to confirm homologous recombination events.

Selected cells can be injected into a blastocyst (or other stage of development suitable for the purposes of creating a viable animal, such as, for example, a morula) of an animal (e.g., a mouse) to form chimeras (see e.g., Bradley, A. in Teratocarcinomas and Embryonic Stem Cells: A Practical Approach, E. J. Robertson, ea., IRE, Oxford, pp. 113-152 (1987)). Alternatively, selected ES cells can be allowed to aggregate with dissociated mouse embryo cells to form an aggregation chimera. A chimeric embryo can then be implanted into a suitable pseudopregnant female foster animal and the embryo brought to term. Chimeric progeny harboring the homologous recombined DNA in their germ cells can be used to breed animals in which all cells of the animal contain the homologously recombined DNA.

In addition to the above described methods of inactivation of endogenous loci, additional preferred methods of inactivation are available and may include for example, use of the tet transcription system to utilize temporal control of luciferase (Proc. Natl. Acad. Sci. 91:9302-9306 (1994)) or introduction of deoxycycline transcriptional regulatory controls for tissue specific control (Proc. Natl. Acad. Sci. 93:10933-10938 (1996)).

An additionally preferred method for functional inactivation includes employment of the cre-lox deletion, site specific recombination system for targeted knock-out of genetic loci, wherein loxP sites are inserted to flank genes of interest and ore recombinase activated to delete genes (Curr. Opin. Biotechnol., 5:521-527 (1994)).

Alternatively, antisense or RNAi methods may be utilized in order to inhibit transcription of a desired gene, thus resulting in functional disruption of endogenous gene (knock-down methods). In such a situation, oligonucleotides are generated which target specific sequences of a gene of interest, wherein successful targeting results in inhibited production of the functional protein. For example an RNAi vector such as pHUSH as described in (U.S. application Ser. No. 11/460,606) could also include the luciferase gene, either expressed together with the target gene via an internal ribosomal entry site (IRES) or under separate promoter control. This would result in a gene of interest being knocked down by the RNAi and the luciferase would track the fate of these cells.

### C. Determining Expression of the Transgene.

Transgenic animals may be screened for the presence and/or expression of the transgene in the desired tissue, cell or animal by any suitable method. Screening is often accomplished by Southern blot or Northern blot analysis, using a probe that is complementary to at least a portion of the transgene. Alternatively, the Rosa26-luciferase transgene can be further verified by PCR analysis of genomic DNA from homozygous offspring. Presence or absence of luciferase mRNA in Rosa26-luciferase mice can be confirmed by PCR amplification of cDNA generated from organs of mice believe to carry the transgene.

Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Because the luciferase transgene is a marker, transgenic animals carrying the transgene can be screened by bioluminescence. For screening large numbers of mice, tail clips can be taken and placed in a solution containing the luciferin substrate. Positive tails will be bioluminescent.

### D. Uses of Transgenic Animals

Transgenic animals of the present invention represent models of cell function in humans. Accordingly, these animals are useful in studying the mechanisms behind cell function and related events, and to generate and test products (e.g., antibodies, small molecules etc.) useful in treating and diagnosing associated human diseases, including cancer and autoimmune conditions.

A transgenic animal of the present invention can further provide an indication of the safety of a particular agent for administration to a human. For example, an agent can be administered to the transgenic animal and any toxic or adverse effects as a result of the administration of the agent to the animal can be monitored or identified as an indication of the safety and tolerability of the agent for in vivo human use. Adverse events that may occur on a short term basis include headache, infection, fever, chills, pain, nausea, asthenia, pharyngitis, diarrhea, rhinitis, infusion reactions, and myalgia. Short term adverse events are measured in days post treatment. Long term adverse effects include cytoxicity of certain cell types, bleeding events due to thrombocytopenia, release of mediators due to inflammatory and/or allergic reactions, inhibition of the immune system and/or development of an anti-therapeutic agent antibody, end organ toxicity, and increased incidence of infection or malignancy. Long term adverse events are measured in months post treatment. The effect of the agent is studied by administration of the agent and the luciferin substrate and either specific cells or the whole body subjected to bioluminescent imaging to look for specific affects.

The transgenic animals of the present invention, including cells or tissues can be utilized as models for diseases. Animals of any species, including, but not limited to, mice, rats, rabbits, guinea pigs, pigs, micro-pigs, goats, and non-human primates, e.g., baboons, monkeys, and chimpanzees may be used to generate disease animal models. These systems may be used in a variety of applications. Such assays may be utilized as part of screening strategies designed to identify agents, such as compounds that are capable of ameliorating disease symptoms. Thus, the animal- and cell-based models may be used to identify drugs, pharmaceuticals, therapies and interventions that may be effective in treating disease.

### E. Imaging of Transgenic Animals

In vivo bioluminescence imaging is a versatile and sensitive tool based on the detection of emitted light from cells or tissues. Bioluminescence has been used to track tumor cells, bacterial and viral infections, gene expression and treatment response in a non-invasive manner. Bioluminescence imaging provides for longitudinal monitoring of a disease course in the same animal, a desirable alternative to analyzing a number of animals at many time points during the course of the disease. The bioluminescence signal is detected with a highly sensitive, intensified CCD camera. The camera is mounted in a light-proof container that provides for anesthesia, mouse platforms and internal lighting.

### EXAMPLES

### Example 1

### Creation and Verification of the Rosa26-luciferase Construct

The Rosa26-luciferase construct was made by cloning the murine Rosa26 promoter as a 1.9 Kb Hind III-Xba I fragment derived from pBROAD3 (InvivoGen, San Diego, Calif.) into a vector containing the 1.7 Kb luciferase gene using convenient restriction sites. A polyadenlyation site was attached to the 3′ end of the luciferase gene for better expression of luciferase.

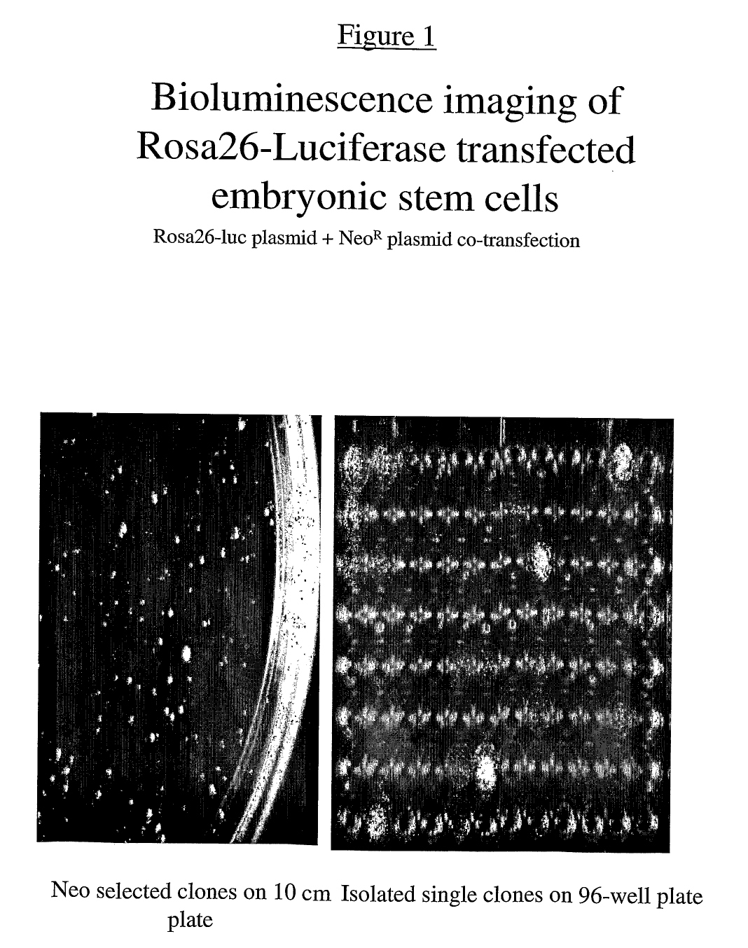

The Rosa26-luciferase construct was co-transfected into ES cells with a Neo resistant plasmid (10:1) and selected in G418. Therefore, the selected clones contain either single NeoR plasmid (luciferase negative) or both plasmids (luciferase positive). Media containing the luciferin substrate was added to the cells which allowed them to be selected directly from the plate (FIG. 1). These clones were then grown into isolated colonies in 96 well plates. An example of this is shown in FIG. 1. Cell number in one well in the 96-well plate is about 50,000 -100,000, and the images were captured with about a one minute exposure.

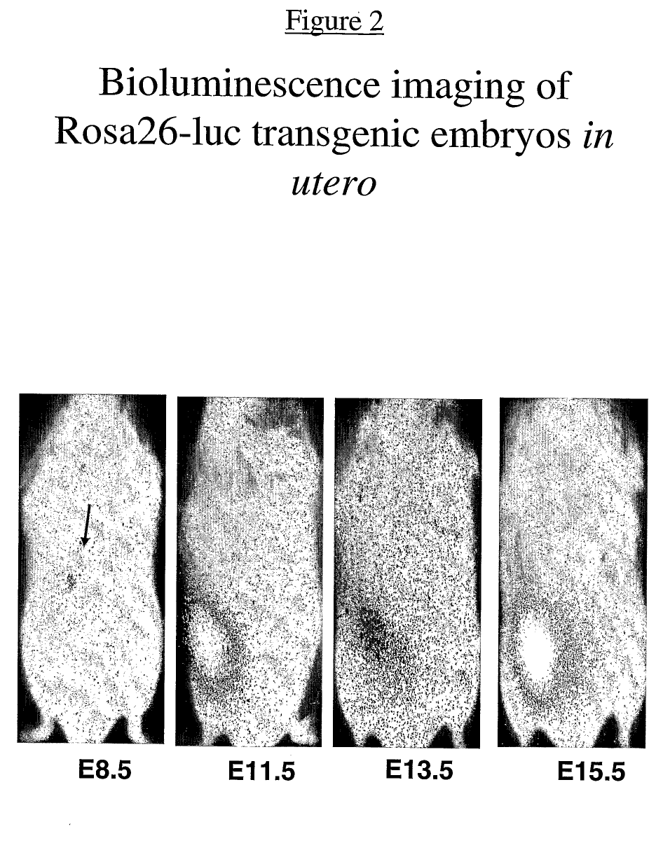

FIG. 2 shows the bioluminescent imaging of Rosa26-luciferase transgenic embryos in utero. The earliest stage at which the developing embryo can be seen is E 8.5. The bioluminescent signal is clearly seen at E11.5, E13.5 and E 15.5. Note the diffuse signal at E13.5, this illustrates that in whole body imaging, the transgenic animal may shift, bringing internal organs over the bioluminescent signal which will momentarily suppress it. This is easily overcome by simply turning the mouse back over.

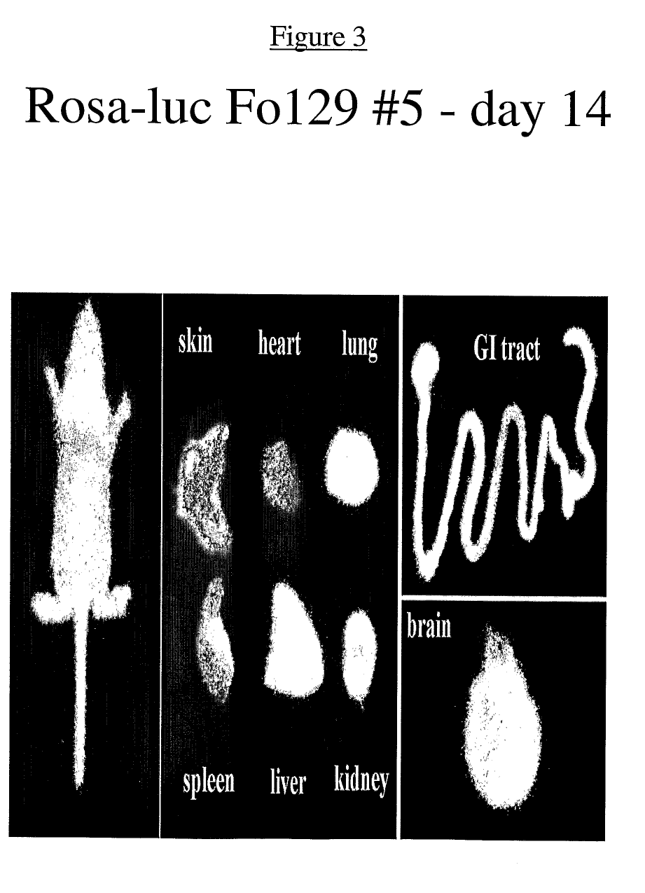

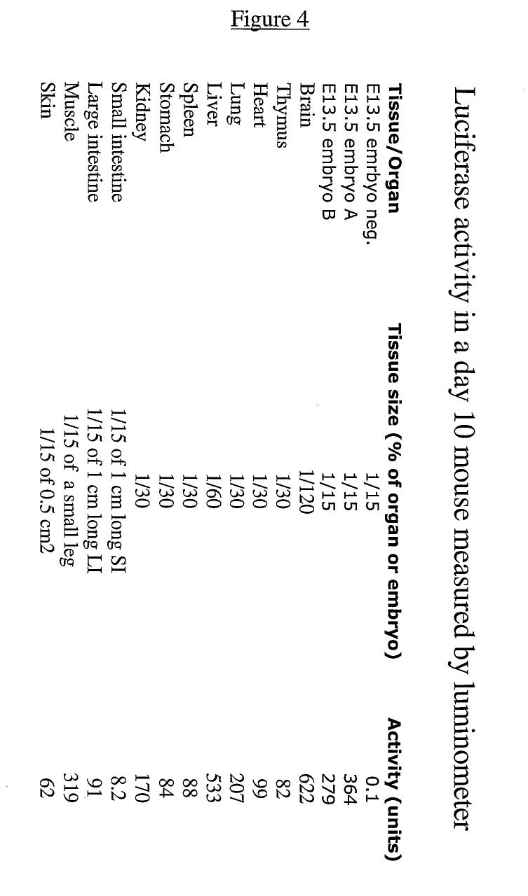

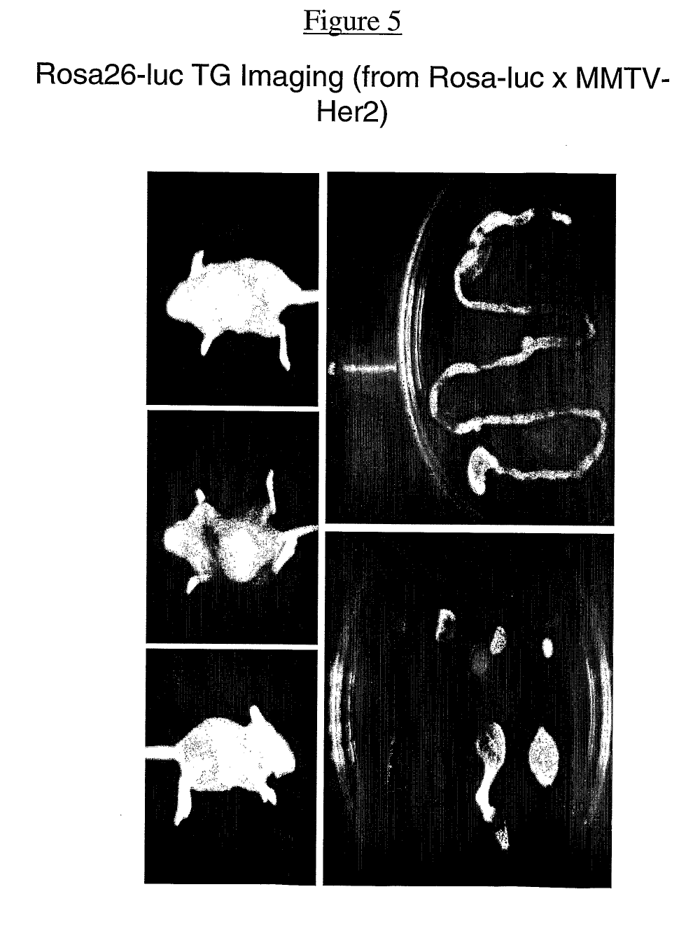

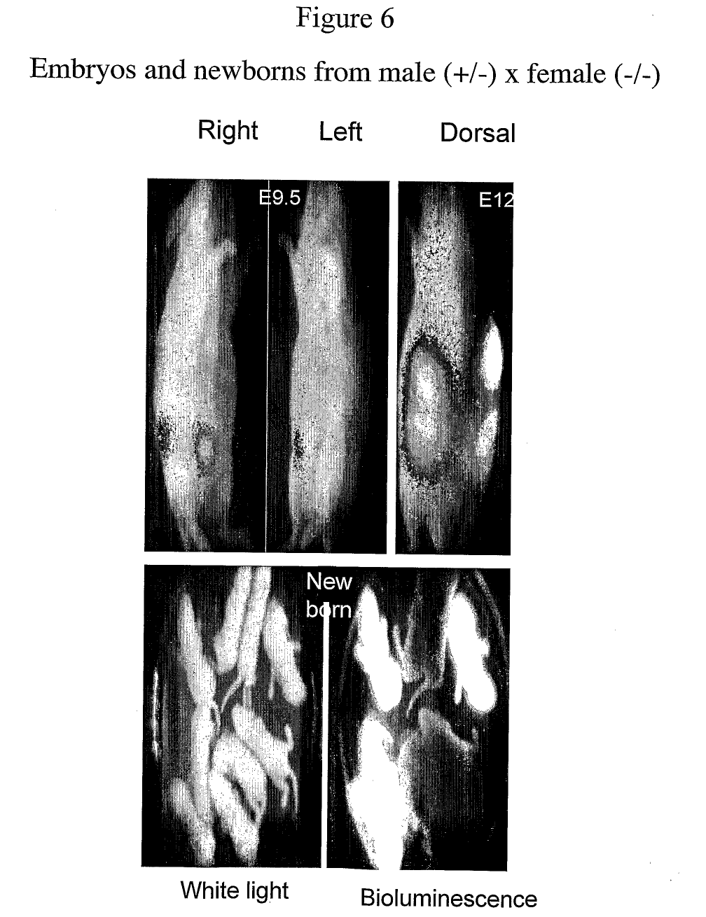

The Rosa26-luciferase construct expresses luciferase strongly in all tissues. FIG. 3 shows the strong signal produced by luciferase via bioluminescent imaging of the whole body of an adult mouse. Examination of the mouse organs showed that the luciferase signal was detected in skin, heart, lung, spleen, liver, kidney, brain and gastrointestinal (GI) tract. This result can be quantified by harvesting the tissue, preparing a protein extract and reading the resulting signal with a luminometer. Transgenic embryos and adult tissues assayed in this manner produced a relative activity which is shown in FIG. 4. Founder Rosa26-luciferase transgenic mice have been monitored for approximately a year now with no observed pathology.

### Example 2

### Repopulation of Blood Lineages Using Marrow from Rosa26-luciferase Trangenics

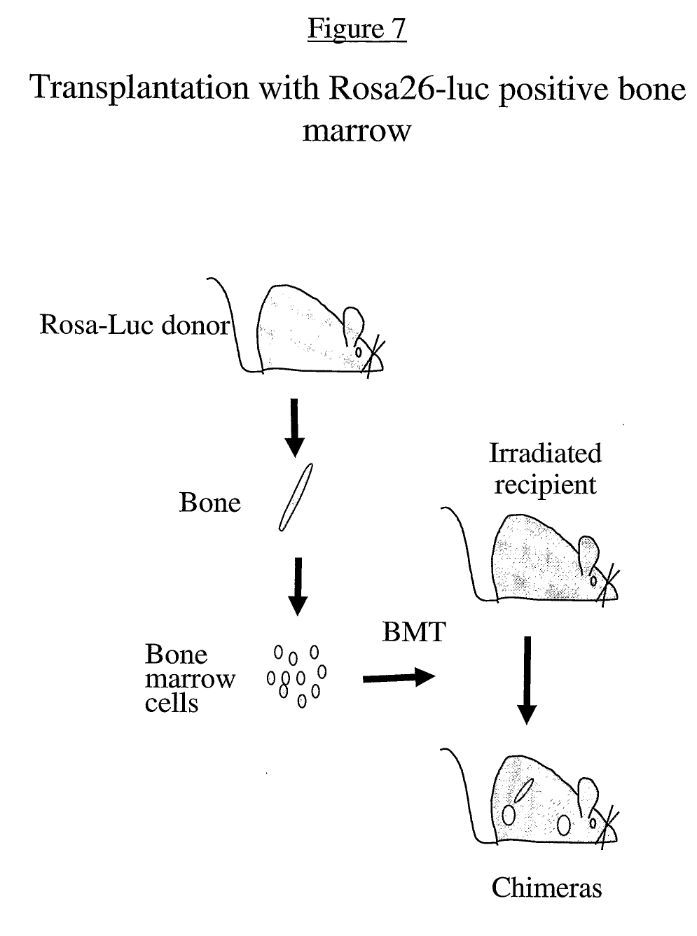

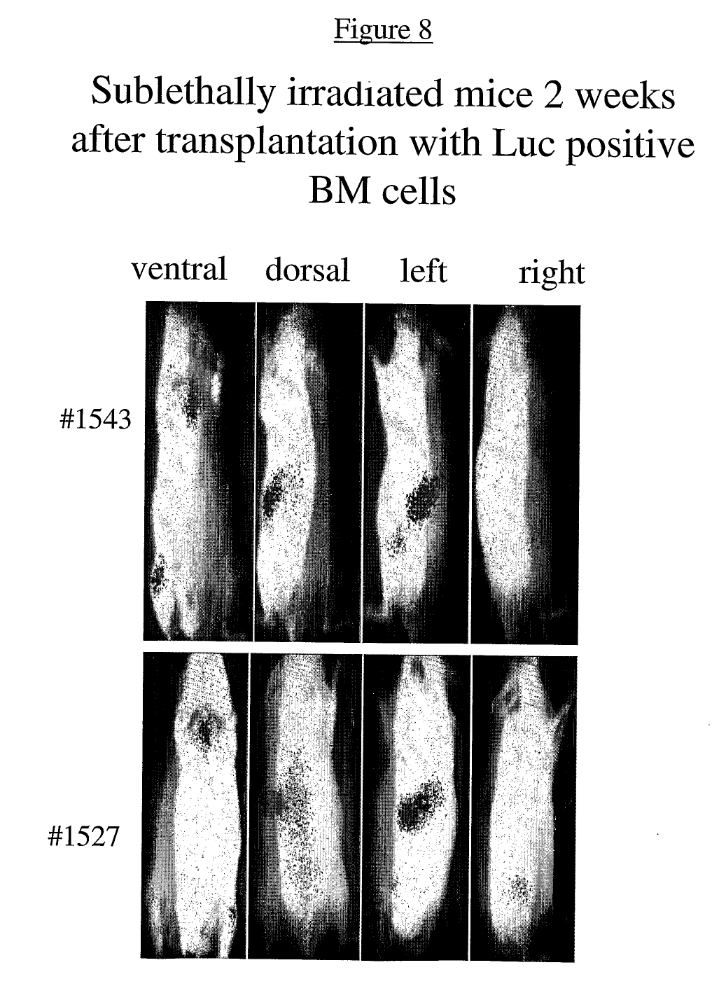

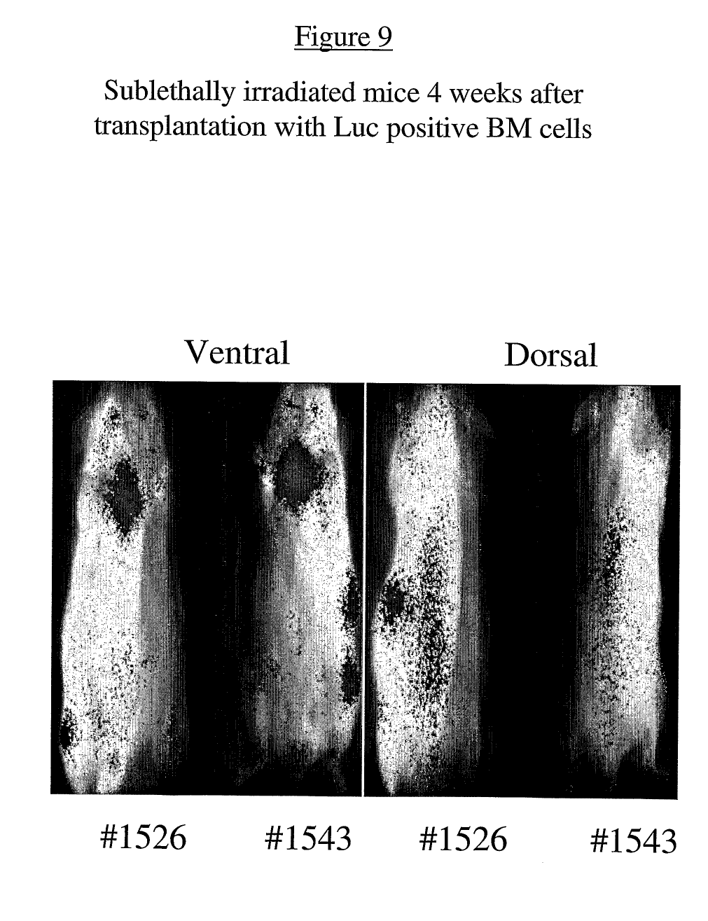

Hematopoietic stem cells (HSCs) found in the bone marrow (BM) are multipotent. The full spectrum of differentiated blood cells (macrophages, megakaryoctes, erythrocytes etc.) have their origins in HSCs. When a mouse is whole body irradiated it becomes cytopenic. The irradiated mouse may die unless BM containing HSCs is used to “repopulate” the mouse with blood cells. The Rosa26-luciferase transgenic mouse was used as a BM donor to whole body irradiated normal mice. Recipients were either sublethally treated with 350 rads or lethally treated with 2×550 rads gamma irradiation. Each recipient was transplanted with about 10⁶ bone marrow cells. Both donors and recipients are in FVB background. The scheme for doing the experiment is shown in FIG. 7. Sublethally irradiated mice two weeks after transplantation are shown in FIG. 8. Because the irradiation is a 350 rad dose, only 10-20% of bone marrow derived cells are from donor based on previous results from others with FVB mice. After 2 weeks, the signal is localized to thymus, spleen and bone marrow in the legs and backbone (FIG. 8). This was confirmed later with dissected organs. After four weeks, the signal was stronger and localized to lymph nodes (FIG. 9).

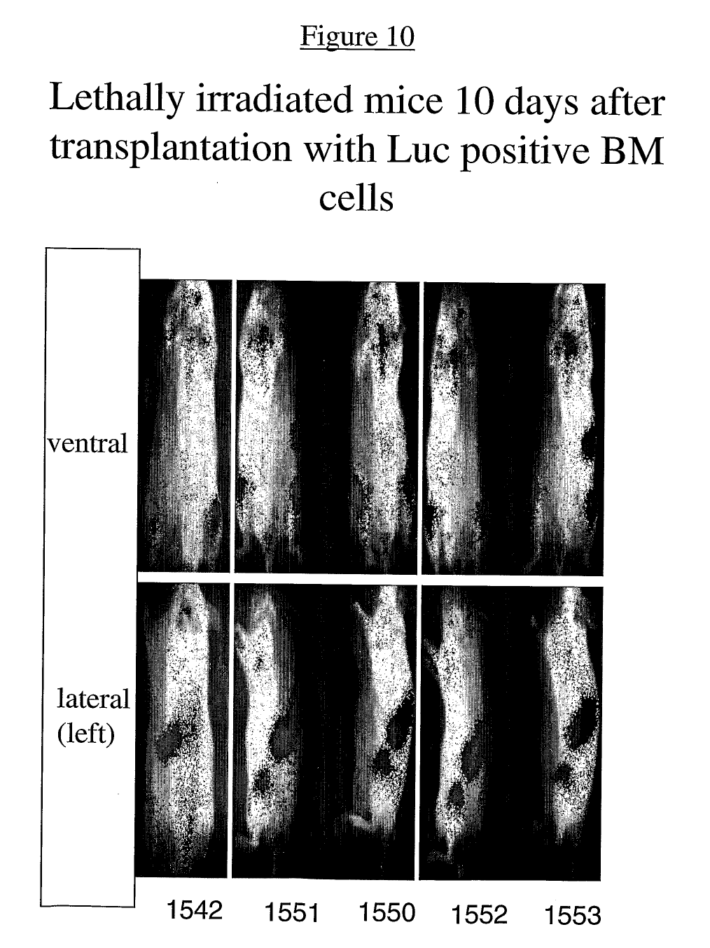

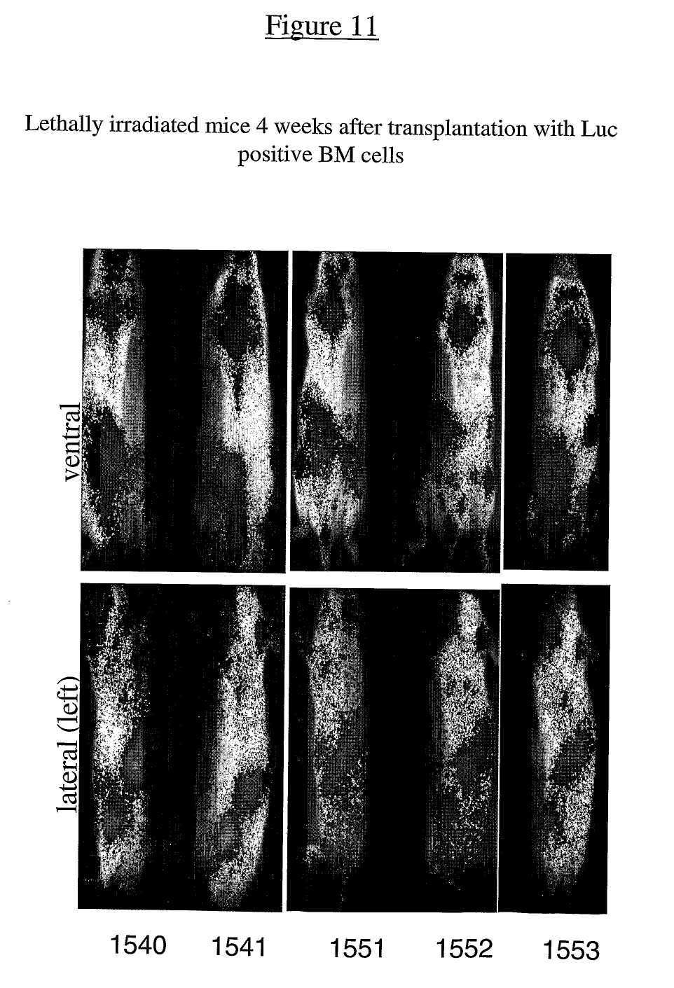

When a much higher dose of radiation is used (2×550 rad dose), 80-90% of BM derived cells will come from donor based on previous results from others with FVB mice. At day ten, the signal is much stronger than sublethally irradiated mice at day fourteen (FIG. 10). Again, the signal is localized to thymus, spleen, lymph nodes and bones and was confirmed later with dissected organs. The mice after four weeks post lethal irradiation, displayed a positive signal in lymph organs such as thymus, spleen, lymph nodes and BM as expected, but also in the skin and gut (FIG. 11). This is consistent with the finding that BM derived cells are involved in tissue repair of skin and gut. The distribution of luciferase positive cells was later confirmed with dissected organs.

### Example 3

### Assay of T cell Populations

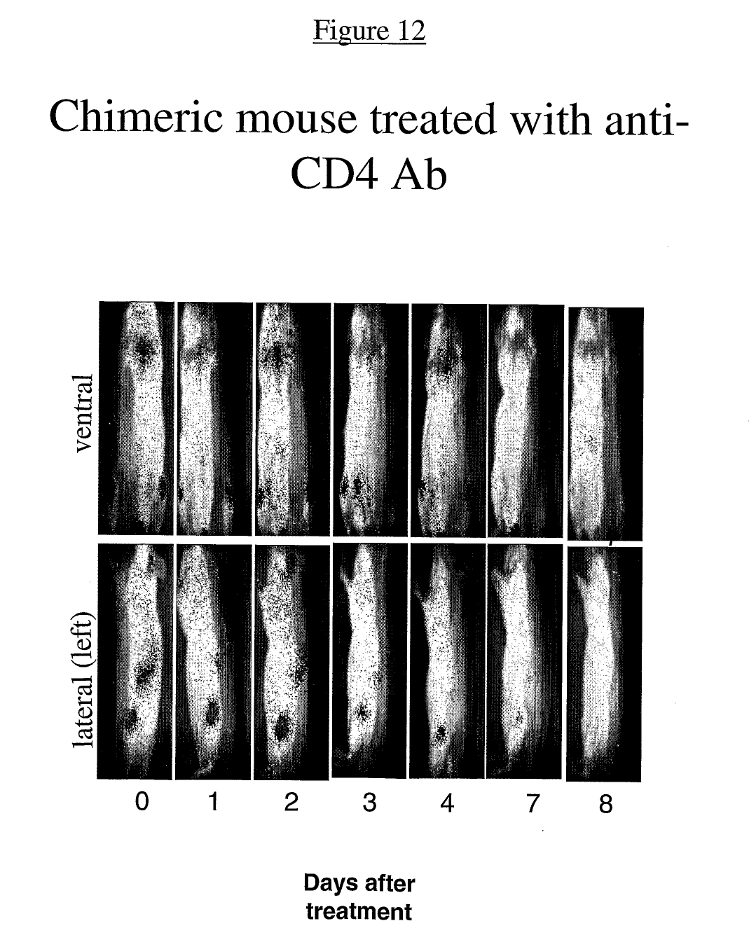

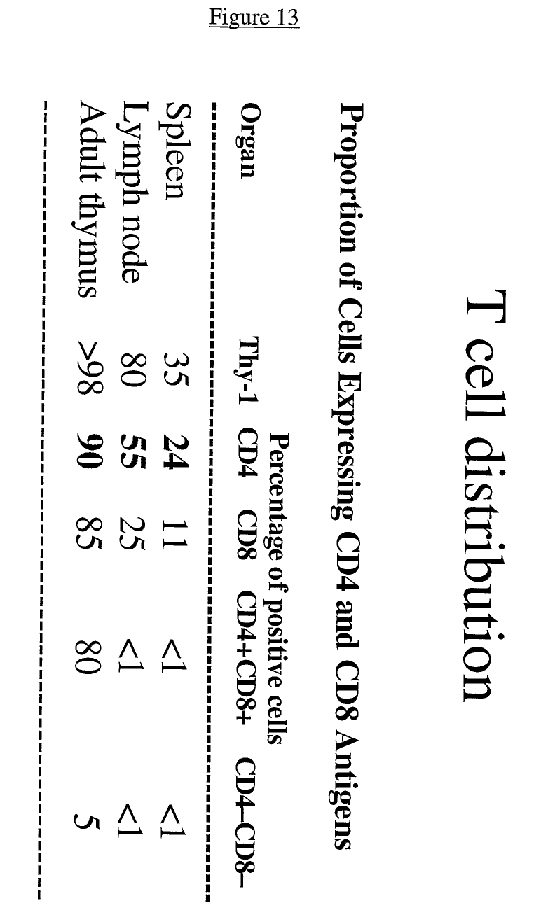

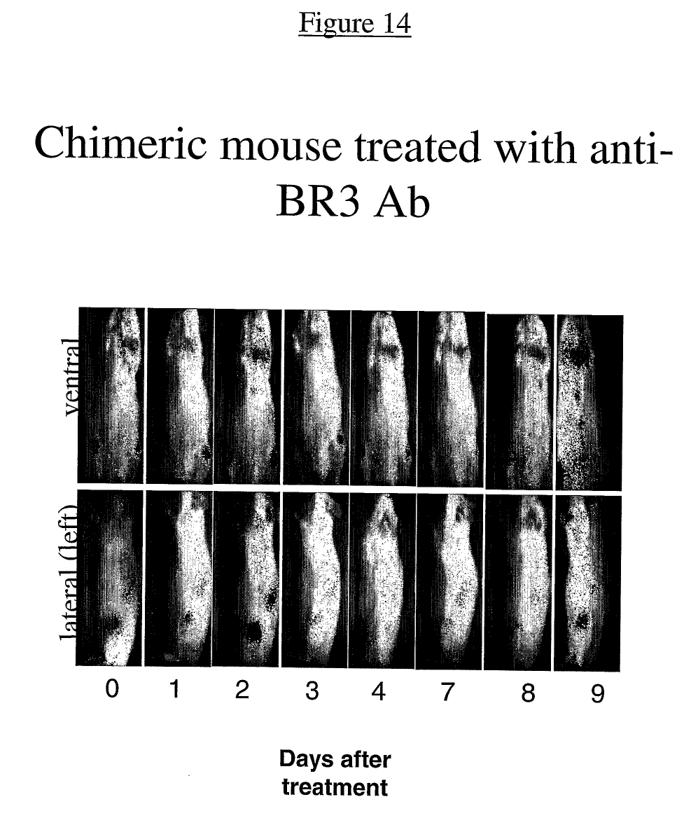

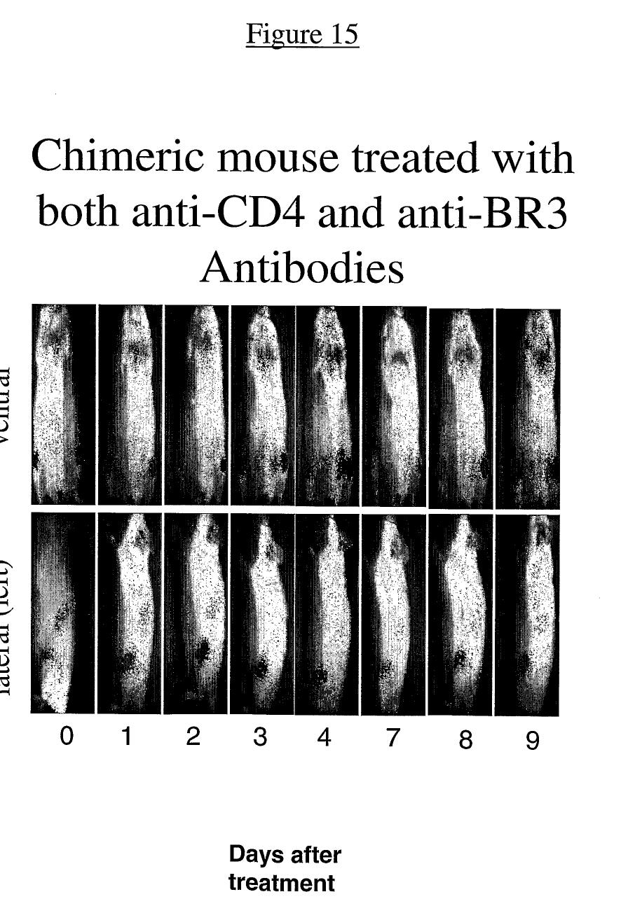

T cell populations were assayed in mice that had been irradiated and transplanted with bone marrow from Rosa26-luciferase mice. Recipient mice were prepared by irradiating 2-3 months old FVB mice either at a sublethal dose of 350 rads or lethal dose of 1100 rads (two treatments of 550 rads divided by 3 hrs apart) by using Cesium 137 source. Bone marrow cells were collected from 2-6 month old Rosa26-luc transgenic mice and were injected through the tail vein into recipients at 15-20 million cells/recipient. The host mice were assayed for bioluminescence one week after bone marrow transplantation and cell engraftment was found. One of the advantages of Rosa26-luciferase models is the ability to track cells over time. Antibodies used to deplete immune cells can be useful in reducing the immune response in autoimmune diseases. The mice transplanted with Rosa26-luciferase BM cells were given a one time intraperitoneal antibody injection of either anti-CD4 or anti-BR3 antibodies at dose of 0.2 mg/mouse. FIG. 12 shows the result of the anti-CD4 injection. Luciferase positive T-cells have accumulated in the thymus, lymph nodes and spleen. This is described in a breakdown of T cell populations as shown in FIG. 13. Treatment with anti-CD4 over an eight day period depletes the number of the T cells found in the thymus, as shown by a loss of bioluminescence (FIG. 12). The host mice treated with anti-BR3 antibody over an eight day period showed loss of B cells in the spleen and bone (FIG. 14). Mice treated with a combination of anti-CD4 antibodies and anti-BR3 antibodies actually showed less efficient immune cell depletion than with individual antibodies (FIG. 15). This model shows that Rosa26-luciferase mice can be used in modeling immune cell depletion for diseases such as Systemic Lupus Erythematosus, rheumatoid arthritis and osteoarthritis.

### Example 4

### Allograft Tumor Models

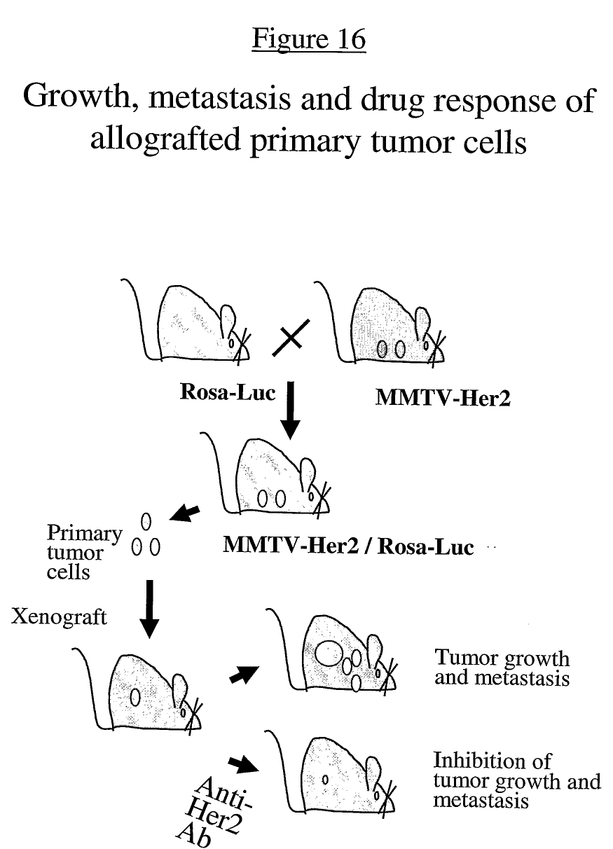

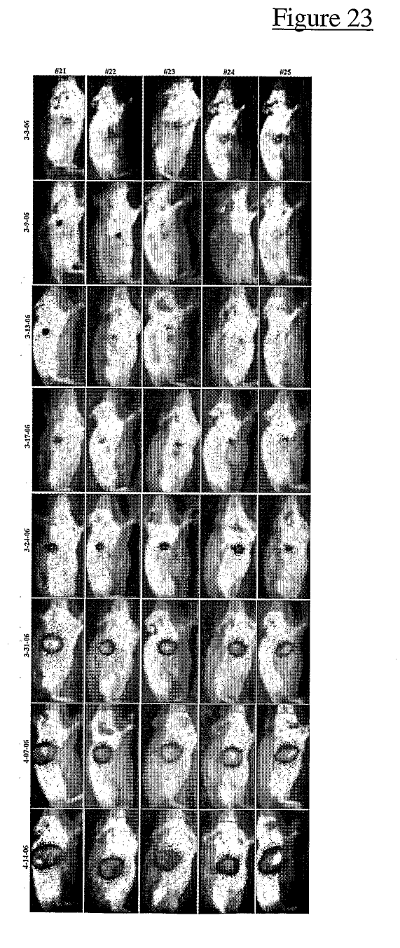

A useful aspect of the Rosa26-luciferase transgenic mice is as a tumor model. Rosa26-luciferase transgenic mice can be crossed with a mouse model of tumorigenesis, such as the MMTV-HER2 transgenic mice (Finkle et al., Clin. Can. Res. 10:2499-2511 (2004)). Female MMTV-HER2 mice develop mammary adenocarcinoma at about 6 months. Lung metastasis is seen in about 23% of the mice. The MMTV-HER2 mice may be crossed with the Rosa26-luciferase mice to create progeny that develop mammary adenocarcinomas expressing luciferase. These adenocarcinomas can then be xenografted into host nude mice and the growth and metastasis of the tumor cells can be monitored without killing the grafted mouse. Such a scheme is depicted in FIG. 16 and was performed. The results are shown in FIG. 23. Two tumors were transplanted in five beige nude mice. Each mouse is shown in a column in FIG. 23. Imaging began 3 days after transplantation and lasted for about 6 weeks, with images taken of each mouse every 4-7 days. In FIG. 23, the rows in descending order depict images taken for each mouse at progressively later time points. At the earlier time points, it was difficult to measure the tumor size but the luciferase signal was quantifiable. At later time points, the intensities of the luciferase signal correlated with tumor size. The tumor sizes in the seventh row from the top are as follows, from left to right: 794, 544, 487, 407, and 442 mm³.

### Example 5

### In vivo Proliferation and Distribution of Specific Immune Cells Treated with Cytokines or in Disease

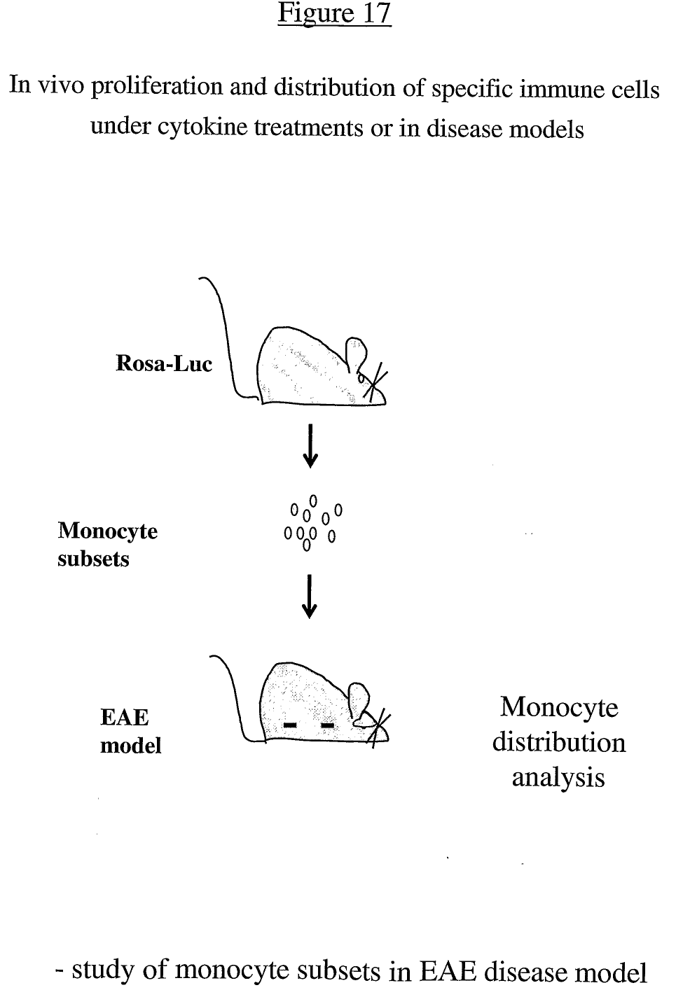

EAE is a T cell mediated autoimmune disease characterized by T cell and mononuclear cell inflammation and subsequent demyelination of axons in the central nervous system. EAE is generally considered to be a relevant animal model for MS in humans (Bolton, C., Multiple Sclerosis 1:143 (1995)). Both acute and relapsing-remitting models have been developed. Agents can be tested for monocyte stimulatory or inhibitory activity against immune mediated demyelinating disease using the protocol described in Current Protocols in Immunology, above, units 15.1 and 15.2. In this model monocytes are isolated from Rosa26-luciferase mice and injected in EAE disease model mice (FIG. 17). EAE mice are often in-bred to reliably produce susceptibility to EAE in the animals. The cells can be tracked as to what regions of body they invade. Agents believed to alleviate EAE can be tested in this model by determining their effect on the monocyte population.

### Example 6

### In vivo Lymphocyte Clearance by Antibodies

T and B cells both comprise cell surface proteins that can be utilized as markers for differentiation and identification. One such human B cell marker is the human B lymphocyte-restricted differentiation antigen Bp35, also known as “CD20.” CD20 is expressed during early pre-B cell development and remains until plasma cell differentiation. It is believed that the CD20 molecule regulates a step in the activation process that is required for cell cycle initiation and differentiation.

CD20 is present on both normal B cells as well as malignant B cells, whose unabated proliferation can lead to B cell lymphoma. Thus, the CD20 surface antigen has the potential of serving as a candidate for targeting of B cell lymphomas with antibodies specific to the antigen. These anti-CD20 antibodies specifically bind to the CD20 cell surface antigen of both normal and malignant B cells, leading to the destruction and depletion of B cells. Chemical agents or radioactive labels having the potential to destroy the tumor can be conjugated to the anti-CD20 antibody such that the agent is specifically delivered to the neoplastic B cell.

The use of monoclonal antibodies targeting CD20 has been described (see, for example, Weiner, Semin. Oncol., 26, 43-51 (1999); Gopal and Press, J. Lab. Clin. Med., 134, 445-450 (1999); White et al., Pharm. Sci. Technol. Today, 2, 95 101 (1999)). Rituxan™ is a chimeric anti-CD20 monoclonal antibody that has been used widely both as a single agent and together with chemotherapy in patients with newly diagnosed and relapsed lymphomas (Davis et al, J. Clin. Oncol., 17, 1851 1857 (1999); Solal-Celigny et al., Blood, 94, abstract 2802 (1999); Foran et al., J. Clin. Oncol., 18, 317-324 (2000). The use of radiolabeled antibody conjugates has also been described (for example, Bexxar; Zelenetz et al., Blood, 94, abstract 2806 (1999)).

The use of antibodies targeting CD20 has also been described for other conditions, especially those involving autoimmunity. For example, anti-CD20 antibody therapy has been or is being evaluated in treatment of rheumatoid arthritis, systemic lupus erythromatosis and ankylosing spondylitis. (Protheroe et al., Rheumatology 38:1150(1999)). Other autoimmune conditions have also been investigated for the treatment with anti-CD20 antibodies leading to B cell depletion such as autoimmune thrombocytopenia and neutropenia, and autoimmune hemolytic anemia. (Trape et al., Haematologica 88:223 (2003); Arzo et al., Annals of Rheumatic Diseases 61:922 (2002)).

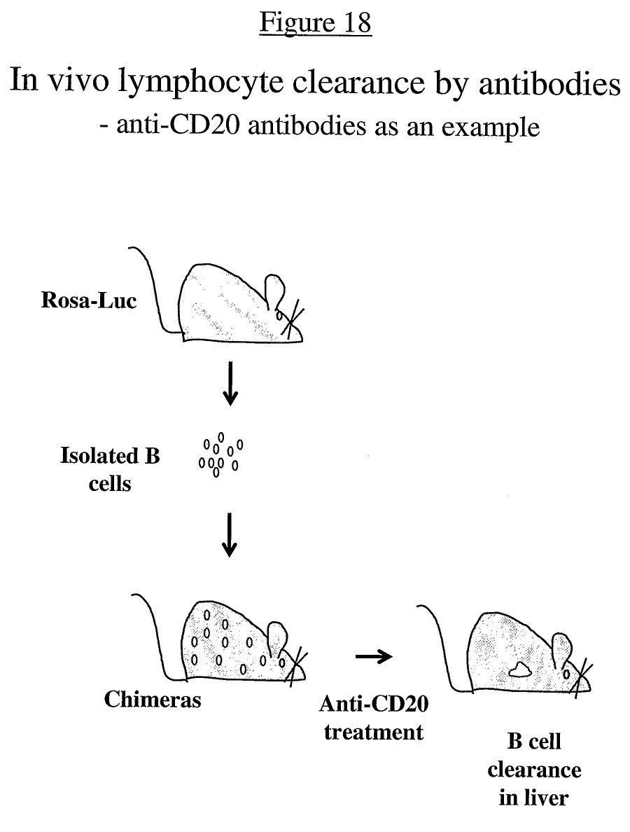

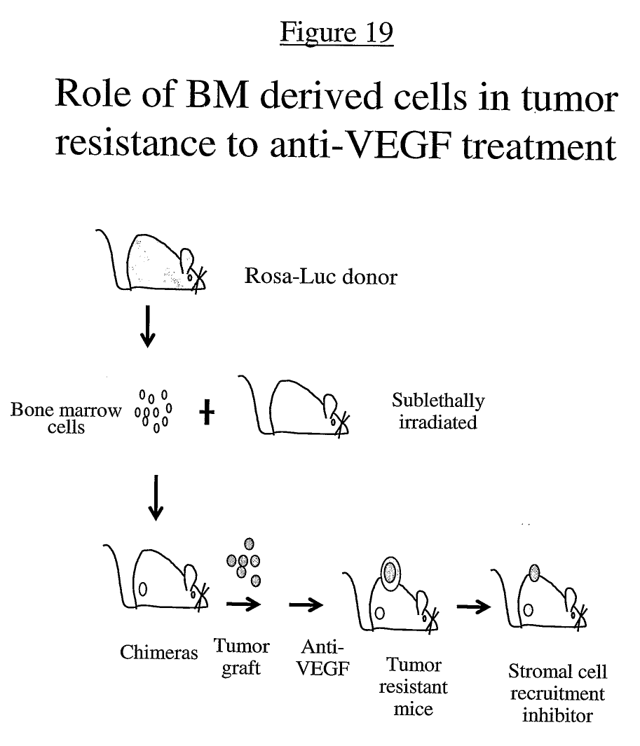

The Rosa26-luciferase mice can be used to study the efficacy of an agent that would cause immune cell clearance (FIG. 19). Immune cells can be isolated from the Rosa26-luciferase mice and injected into normal mice. The normal mice can then be treated with an agent that is predicted to deplete or stimulate the immune cells. For example, B cells isolated from the Rosa26-luciferase mice, injected into normal mice, and treated with an anti-CD20 antibody (FIG. 18). The treated mice can be assayed for the clearance of the luciferase positive B cells and determine how effective the agent is. Because the mice do not need to be sacrificed, many timepoints can be taken to determine if the agent is fast or slow acting.

### Example 7

### Bone Marrow Cells Replenish Other Tissues

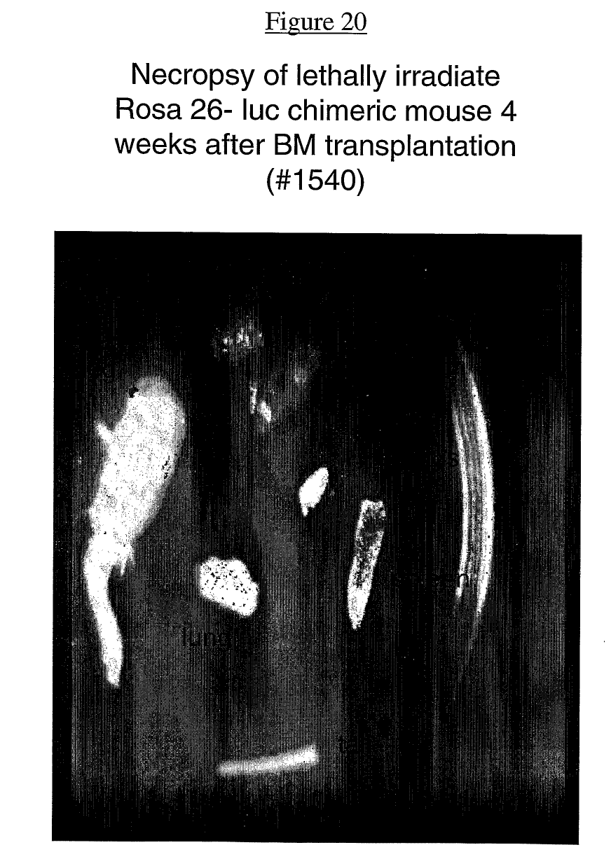

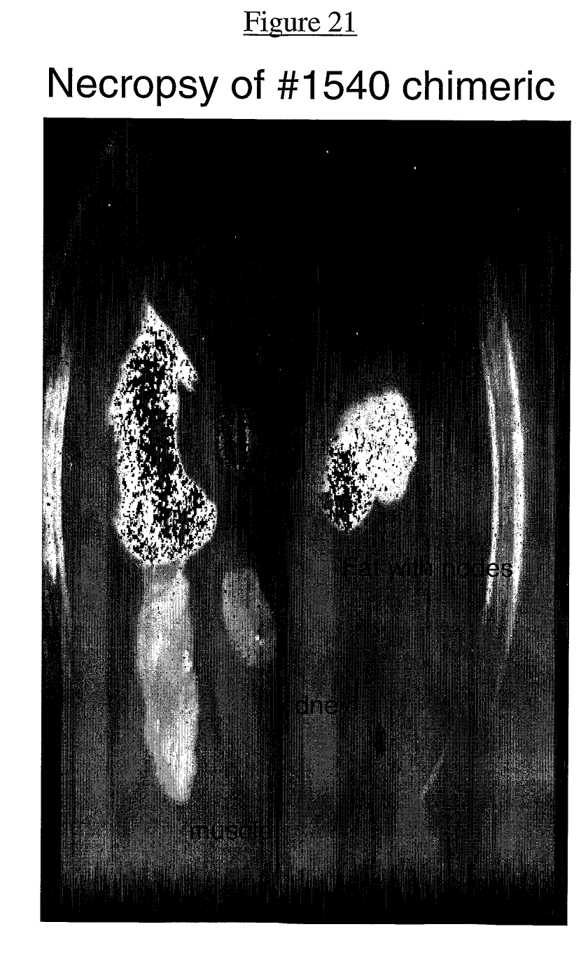

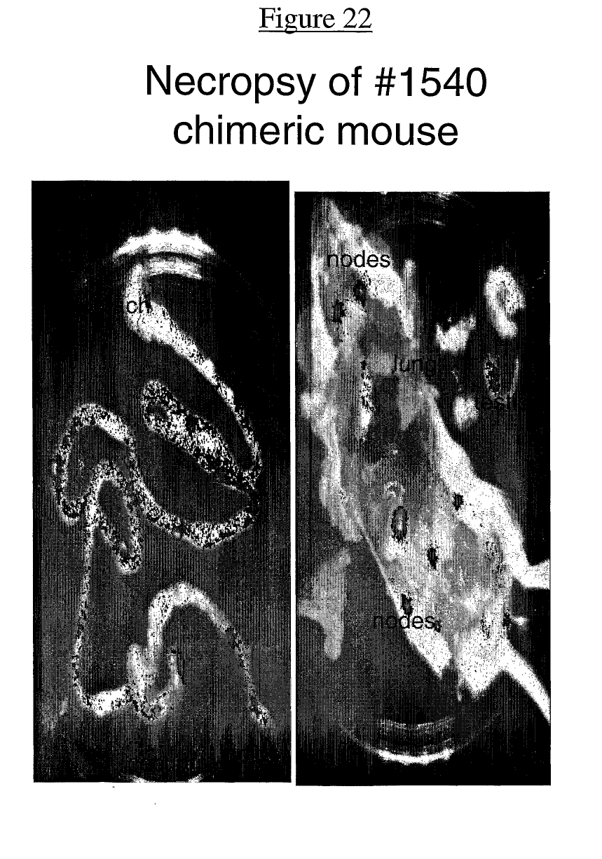

Mice were lethally irradiated and transplanted with bone marrow cells from Rosa26-luciferase mice. Five mice were whole body imaged to show the areas where the transplanted cells had migrated to (FIG. 11). A mouse was sacrificed to determine if the pluripotent bone marrow cells had migrated to other tissues and added to their cell population. FIG. 20-22 shows that cells transplanted cells had incorporated themselves to varying degrees into heart, liver, thymus, spleen, muscle, kidney, testis, colon, skin and body fat.

### Example 8

### Bioluminesence Imaging

In order to overcome the challenges associated with imaging the low signal photon flux that results from bioluminescent emission localized within a living mouse, a dual-stage microchannel plate (MCP) intensified, cooled CCD camera (ICCD) is used as the imaging system. Cooling of the high quantum efficiency GaAsP photocathode virtually eliminates the dark counts that are typically a limitation for this type of application. The dual stage MCP provides light gains of up to 1 million thus amplifying the incident photon flux signal to well above the read noise of the CCD. Combined with a dedicated software system, this configuration can image challenging disease models with low abundance transgene expression, and it can reduce acquisition times that improve the utility of bioluminesence as a screening tool.

Luciferase may be incorporated into mice either as a transgene or via injection of a cell line that has been transfected to express luciferase (e.g. xenograft studies). Luciferase-bearing mice receive an intraperitoneal injection of the luciferase substrate luciferin. They are imaged by placing them in a light-tight imaging chamber which incorporates the ICCD. First, a reference image of the mouse is acquired, followed by a bioluminescence image about 5 minutes after the luciferin administration. The exposure time of the camera is set such that it is able to localize the bioluminescence emission from the mouse (long exposure for weak signal). Typically, an exposure of a few seconds is used. Data is processed by superimposing the bioluminescence image onto the reference image. The signal may be quantitated using a region of interest analysis of pixel intensity in the bioluminescence data image.

What is claimed is:

1. A transgenic animal comprising a heterologous construct, said heterologous construct comprising a nucleotide sequence encoding luciferase operably linked to a Rosa26 promoter, wherein said heterologous construct is integrated randomly in the transgenic animal's genome.

2. The transgenic animal of claim 1, wherein the Rosa26 promoter comprises the nucleotide sequence of SEQ ID NO:1.

3. The transgenic animal of claim 1 where all cells of the transgenic animal express luciferase.

4. The transgenic animal of claim 3 wherein the animal is fertile and transmits the luciferase transgene to its offspring.

5. The transgenic animal of claim 4, wherein the animal is heterozygous for the luciferase transgene.

6. The transgenic animal of claim 4, wherein the animal is homozygous for the luciferase transgene.

7. A method of monitoring growth of a tumor comprising:
a) breeding a transgenic animal expressing luciferase with another transgenic animal that produces tumors to generate offspring;
b) grafting luciferase positive tumor cells from the offspring into a host animal; and
c) imaging bioluminescence from the host animal.

8. A method of identifying an agent capable of reducing tumor cell proliferation comprising:
a) breeding a transgenic animal expressing luciferase with another transgenic animal that produces tumors to generate offspring;
b) grafting luciferase positive tumor cells from the offspring into a host animal;
c) administering the agent to the host animal;
d) imaging the host animal and assaying for the reduction of tumor cells.

9. A method of identifying an agent capable of depleting or killing immune cells comprising:
a) isolating luciferase positive immune cells from a transgenic animal expressing luciferase;
b) transplanting the immune cells into a host animal;
c) administering the agent to the host animal;
d) imaging the host animal and assaying for reduction of immune cells.

10. The method of claim 9, wherein the host animal is susceptible to EAE.

11. The method of claim 9, wherein the immune cells are lymphoma cells.

12. A method of imaging comprising:
a) creating a transgenic animal as in claim 1;
b) injecting the transgenic animal with an amount of luciferin substrate effective to generate bioluminescence;
c) detecting bioluminescence from the transgenic animal.


SEQUENCE LISTING (1 sequence)

SEQ ID NO: 1 - DNA, 1932 bp, Mus musculus
gatctcgacttaattaacctgcaggtgaagacgttacacaagtaacatgagaaagcagaaaatgcaggtcatccacgcacccctgacccaggccagcagggcgggctgcagcatcagtacacaggagaaagatccttattcctaagaatgagaaaggcaaaggcgcccgatagaataaattagcatagaaggggctttcccaggagttaaaactttccttctgagcgattacctactaaaaccagggcttttgcccactaccatttacctaggatcttggcttgcacggattcataggggcatatccctccccctcttctttagagtcgttcttaaaagatcgctctccacgccctaggcagggaaaacgacaaaatctggctcaattccaggctagaaccctacaaattcaacagggatatcgcaaggatactggggcatacgccacagggagtccaagaatgtgaggtgggggtggcgaaggtaatgtctttggtgtgggaaaagcagcagccatctgagataggaactggaaaaccagaggagaggcgttcaggaagattatggaggggaggactgggcccccacgagcgaccagagttgtcacaaggccgcaagaacaggggaggtggggggctcagggacagaaaaaaaagtatgtgtattttgagagcagggttgggaggcctctcctgaaaagggtataaacgtggagtaggcaatacccaggcaaaaaggggagaccagagtagggggaggggaagagtcctgacccagggaagacattaaaaaggtagtggggtcgactagatgaaggagagcctttctctctgggcaagagcggtgcaatggtgtgtaaaggtagctgagaagacgaaaagggcaagcatcttcctgctaccaggctggggaggcccaggcccacgaccccgaggagagggaacgcagggagactgaggtgacccttctttcccccggggcccggtcgtgtggttcggtgtctcttttctgttggacccttaccttgacccaggcgctgccggggcctgggcccgggctgcggcgcacggcactcccgggaggcagcgagactcgagttaggcccaacgcggcgccacggcgtttcctggccgggaatggcccgtacccgtgaggtgggggtggggggcagaaaaggcggagcgagcccgaggcggggagggggagggccaggggcggagggggccggcactactgtgttggcggactggcgggactagggctgcgtgagtctctgagcgcaggcgggcggcggccgcccctcccccggcggcggcagcggcggcagcggcggcagctcactcagcccgctgcccgagcggaaacgccactgaccgcacggggattcccagtgccggcgccaggggcacgcgggacacgccccctcccgccgcgccattggcctctccgcccaccgccccacacttattggccggtgcgccgccaatcagcggaggctgccggggccgcctaaagaagaggctgtgctttggggctccggctcctcagagagcctcggctaggtaggggatcgggactctggcgggagggcggcttggtgcgtttgcggggatgggcggccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaattgtactaaccttcttctctttcctctcctgacaggtgtgaaacaggaagagaa